(19) United States
(12) Patent Application Publication
(10) Pub. No.: US 2005/0159333 A1
(43) Pub. Date: Jul. 21, 2005

(54) Methods of modulating smooth muscle contractility

(76) Inventors: Zunxuan Chen, King of Prussia, PA (US); Erding Hu, King of Prussia, PA (US); Timothy D. Westfall, King of Prussia, PA (US); Alexandria Wibberley, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/513,139

(22) PCT Filed: Apr. 30, 2003
(86) PCT No.: PCT/US03/13385

(30) Foreign Application Priority Data
May 2, 2002 (US) 60377504

(51) Int. Cl. A61K 38/17; A61N 1/00

(52) U.S. Cl. 514/2; 607/2

(57) ABSTRACT

Method for modulating bladder smooth muscle contractility comprising the step of contacting a polypeptide in a ROCK pathway with a compound that modulates an activity of said polypeptide.

### FIELD OF THE INVENTION

This invention relates to newly identified methods for modulating smooth muscle contractility, particularly for the treatment of lower urinary tract disorders and overactive bladder, especially those disorders of humans.

### BACKGROUND OF THE INVENTION

It is widely accepted that the key signal to activate the contractile apparatus in smooth muscle is an increase in intracellular calcium concentration ([Ca²⁺]ᵢ). This promotes the binding of Ca²⁺ to calmodulin (herein “CaM”), the resultant Ca²⁺-CaM complex activates myosin light chain kinase (MLCK) to phosphorylate the myosin light chain (herein “MLC”) of myosin and smooth muscle contraction occurs (for review see Horowitz, et al., Physiological Reviews., 76(4), 967-1003 (1996). However, recently, secondary mechanisms have been identified that can modulate, independently of Ca²⁺, smooth muscle contractility. Activation of excitatory receptors coupled to G proteins can cause contraction of smooth muscle without necessarily changing [Ca²⁺]ᵢ a process termed ‘Ca²⁺-sensitization’. The small GTPase Rho and one of its downstream effectors, Rho-associated kinase (herein “Rho-kinase” and “ROCK”) have been shown possess important roles in this pathway. Activated ROCK phosphorylates, and thus inactivates, smooth muscle myosin phosphatase, preventing the dephosphorylation of MLC by this enzyme and thus sensitizing the smooth muscle contractile machinery to Ca²⁺ (for review see, Somylo, a. p. & Somylo, a. v., Journal of Physiology, 522, 177-185 (2000).

The use of a specific inhibitor of ROCK, Y-27632 (Uehata, et al., Nature, 389, 990-994 (1997); Davies, et al., Biochemical Journal, 351, 95-105 (2000); Ishizaki, et al., Molecular Pharmacology, 57, 976-983 (2000)), has demonstrated a role for this enzyme in Ca²⁺-independent regulation of contraction in a number of tissues, including vascular (Uehata, et al., Nature, 389, 990-994 (1997)), airway (Iikuka et al., European Journal of Pharmacology, 406, 273-279 (2000); Yamagata et al., Pulmonary Pharmacology and Therapeutics, 13, 25-29 (2000) and genital (Chitaley et al., Nature Medicine, 7(1), 119-122, (2001); Rees et al., British Journal of Pharmacology, 133, 455-458 (2001) smooth muscles. Furthermore, recently, Jezior et al. British Journal of Pharmacology, 134, 78-87 (2001) have shown that Y-27632 attenuates bethanechol-evoked contractions in isolated rabbit urinary bladder smooth muscle. However, prior to the instant invention expression and roles of ROCK in urinary bladder smooth muscle was unknown, leaving an unmet medical need, viz., new treatments for lower urinary tract disorders and/or overactive bladder.

### SUMMARY OF THE INVENTION

The present invention relates to methods of modulating smooth muscle contractility via modulating an activity in a ROCK-mediated pathway. Such methods are of interest in relation to methods of treatment of certain diseases, including, but not limited to, urinary tract disorders, particularly lower urinary tract disorders and/or overactive bladder, herein referred to as “diseases of the invention” or “diseases”. In a further aspect, the invention relates to methods for identifying agonists and antagonists (e.g., inhibitors) using the materials or methods provided by the invention, and treating conditions associated with an imbalance in a ROCK-mediated pathway correlating to a disease with an identified compound or other compound capable of affecting such treatment. In a still further aspect, the invention relates to diagnostic assays for detecting diseases associated with inappropriate activity or levels in a ROCK-mediated pathway.

The invention further provides a method for modulating bladder smooth muscle activity comprising the step of contacting a polypeptide in a ROCK pathway with a compound that modulates an activity of said polypeptide.

A further method is provided wherein said polypeptide is ROCK 1 or ROCK 2 or both.

Another method is provided wherein said activity of said polypeptide is selected from the group consisting of: a response to carbachol, a response to neurokinin A, a response to a P2X receptor antagonist, and a response to electrical stimulus.

The invention also provides a method wherein said response is bladder contraction.

A method is also provided wherein said bladder smooth muscle activity is contractility.

The invention also provides a method of treating a mammal for a lower urinary tract disorder or overactive bladder comprising the step of modulating bladder smooth muscle activity.

A further method of treating a mammal for a lower urinary tract disorder or overactive bladder comprising contacting said mammal with a compound that modulates an activity of a polypeptide in a ROCK pathway is also provided.

A still further method is provided wherein said bladder smooth muscle activity is related to expression or activity of ROCK 1 or ROCK 2 or both.

A method wherein said activity of ROCK 1 or ROCK 2 or both is selected from the group consisting of: a response to carbachol, a response to neurokinin A, a response to a P2X receptor antagonist, and a response to electrical stimulus is also provided by the invention.

### BRIEF DESCRIPTION OF THE FIGURES

FIG. 1 graphically illustrates expression of ROCK I and ROCK II in male rat tissues. ROCK I or ROCK II specific message was normalized to GAPDH specific message and the relative amount of mRNA of each sample was compared to that of heart while arbitrarily setting heart mRNA level as 1.0 unit. Each bar represents the mean value of 4 samples and vertical bars show s.e.mean

FIG. 2 graphically illustrates effects of 10 μM Y-27632 and vehicle on carbachol-evoked contractions of the isolated male rat urinary bladder; which are typical traces and data in. Each point represents the mean value of 4-6 experiments and vertical bars show s.e.mean. * P<0.05 compared to vehicle.

FIG. 3 graphically illustrates effects of 10 μM Y-27632 and vehicle on carbachol-evoked contractions of the isolated male rat urinary bladder which are carbachol-evoked responses expressed as percentages of responses evoked by 100 mM KCl. Each point represents the mean value of 4-6 experiments and vertical bars show s.e.mean. * P<0.05 compared to vehicle.

FIG. 4 graphically illustrates effects of 10 μM Y-27632 and vehicle on NKA-evoked contractions of the isolated male rat urinary bladder which are typical traces and data in. Each point represents the mean value of 4-5 experiments and vertical bars show s.e.mean. * P<0.05 compared to vehicle.

FIG. 5 graphically illustrates effects of 10 μM Y-27632 and vehicle on NKA-evoked contractions of the isolated male rat urinary bladder which are NKA-evoked responses expressed as percentages of responses evoked by 100 mM KCl. Each point represents the mean value of 4-5 experiments and vertical bars show s.e.mean. * P<0.05 compared to vehicle.

FIG. 6 graphically illustrates effects of 10 μM Y-27632 and vehicle on KCl-evoked contractions of the isolated male rat urinary bladder which are typical traces and data in. Each point represents the mean value of 5 experiments and vertical bars show s.e.mean.

FIG. 7 graphically illustrates effects of 10 μM Y-27632 and vehicle on KCl-evoked contractions of the isolated male rat urinary bladder which are KCl-evoked responses expressed as percentages of responses evoked by 100 mM KCl (the maximal response). Each point represents the mean value of 5 experiments and vertical bars show s.e.mean.

FIG. 8 graphically illustrates effects of 10 μM Y-27632, 10 μM HA-1077 and vehicle on 10 μM α,β-methylene ATP-evoked contractions of the isolated male rat urinary bladder which are typical traces of responses to α,β-methylene ATP following incubation with antagonist or vehicle and data in. Each bar represents the mean value of 4-6 experiments and vertical bars show s.e.mean. Shaded circles indicate addition of 10 μM α,β-methylene ATP to the tissue strips. * P<0.05 compared to vehicle.

FIG. 9 graphically illustrates effects of 10 μM Y-27632, 10 μM HA-1077 and vehicle on 10 μM α,β-methylene ATP-evoked contractions of the isolated male rat urinary bladder which are responses following incubation with antagonist or vehicle expressed as percentages of control responses. Each bar represents the mean value of 4-6 experiments and vertical bars show s.e.mean. Shaded circles indicate addition of 10 μM α,β-methylene ATP to the tissue strips. * P<0.05 compared to vehicle.

FIG. 10 graphically illustrates effects of 10 μM Y-27632 on electrically-evoked contractions of the isolated male rat urinary bladder. (A) shows a typical individual electrically-evoked bladder contraction at a frequency of 16 Hz indicating the biphasic response and traces in (B) show the effects of 10 μM Y-27632 on electrically-evoked bladder contractions at all frequencies of stimulation. Data in (C) are percentage changes in the amplitude (a) and area under the curve (b) of electrically-evoked contractions following incubation with Y-27632 or vehicle. Each point represents the mean value of 4 experiments and vertical bars show s.e.mean. * P<0.05 compared to vehicle.

FIG. 11 graphically illustrates effects of various combinations of 10 μM Y-27632, 1 μM atropine and 10 μM α,β-methylene ATP on electrically-evoked contractions of the isolated male rat urinary bladder. Data are expressed as percentage changes in the amplitude (a) and area under the curve (b) of electrically-evoked contractions following incubation with Y-27632 and/or atropine (A) or Y-27632 and/or α,β-methylene ATP (B) or vehicle. Each point represents the mean value of 4-5 experiments and vertical bars show s.e.mean. * P<0.05 for effects of a single antagonist compared to vehicle, # P<0.05 for effects of Y-27632 compared with atropine or α,β-methylene ATP alone and ## P<0.05 for effects of combinations of Y-27632 with atropine or α,β-methylene ATP compared with the effects of atropine or α,β-methylene ATP alone.

FIG. 12 graphically illustrates effects of 10 μM Y-27632, 10 μM HA-1077 and vehicle on baseline bladder tensions in the isolated male rat urinary bladder. Data in (A) are baseline bladder tensions (g) following incubation with Y-27632, HA-1077 or vehicle for 45 min measured every 15 min and data in (B) are changes in baseline bladder tensions expressed as percentages of pre-treatment tensions. Each point represents the mean value and vertical bars show s.e.mean of 5-25 experiments.
* P<0.05 compared to vehicle.

### DESCRIPTION OF THE INVENTION

Based on an unmet medical need of providing new treatments for lower urinary tract disorders and overactive bladder, a study was commenced that investigated the expression of ROCK in male rat tissues, including the urinary bladder, and evaluated the effects of ROCK inhibition with Y-27632 on male rat urinary bladder contractility in vitro. The rat was chosen as a well used and accepted model for ROCK studies, providing important information and insights relating to human disease. The present invention was based, in part, on these studies.

Aspects of these studies investigated expression of ROCK in the male rat urinary bladder, and evaluated the effects of ROCK inhibition with Y-27632 on contractility of this tissue in vitro. It was observed that both isoforms of ROCK, ROCK I and ROCK II were detected at high levels in male rat urinary bladder. ROCK II levels were higher in bladder compared to rat aorta, brain, liver, kidney and skeletal muscle (refer to Example 1). Further experiments revealed that Y-27632 (10 μM) significantly attenuated carbachol (58±11% at 3×10⁻⁶ M) and NKA; 69±13% at 10⁻⁶ M-evoked bladder contractions. Furthermore, Y-27632 had no measureable effect on contractile responses to potassium chloride (KCl; 10-100 mM). Surprisingly, Y-27632 (10 μM) significantly attenuated bladder contractile responses to the P2X receptor agonist α,β-methylene ATP (10 μM; 30±7%). Furthermore, fasudil hydrochloride (HA-1077; 10 μM), a ROCK inhibitor, also significantly inhibited α,β-methylene ATP-evoked responses (22±6%). Moreover, Y-27632 (10 μM) significantly attenuated the amplitude and AUC of electrically-evoked bladder contractions (2-16 Hz). Furthermore, the effects of Y-27632 on the amplitude and AUC of responses were not significantly different from the effects of α,β-methylene ATP (10 μM) or atropine (1 μM) alone, respectively. These studies demonstrate the presence of ROCK I and ROCK II in male rat urinary bladder and indicate an involvement of these kinases in G protein-coupled contractile mechanisms in this tissue.

Such studies on which the present invention was, in part, based demonstrated a role for ROCK in G protein-coupled contractile responses of the isolated male rat urinary bladder. This is supported by the observation that Y-27632 attenuated contractions evoked by carbachol and NKA in this tissue. Responses to these agonists in the rat urinary bladder have previously been shown to be mediated by M₃ muscarinic acetylcholine receptors (Longhurst et al., British Journal of Pharmacology, 116, 2279-2285 (1995); Tong et al., Journal of Autonomic Pharmacology, 17, 21-25 (1997)) and tachykinin NK₂ receptors (Hall et al., British Journal of Pharmacology, 107(3), 777-784, (1992)), respectively, which are G protein coupled (for reviews see Eglen et al., Trends in Pharmacological Sciences, 15, 114-119 (1994); Khawaja & Rogers, International Journal of Biochemistry and Cell Biology., 28(7), 721-738 (1996)). Indeed, carbachol and NKA have been shown to increase the production of inositol triphosphate (IP₃) which acts on intracellular Ca²⁺ stores to release Ca²⁺ (Iacovou, et al., Journal of Urology, 144, 775-779 (1990); Harriss, et al., Journal of Urology, 154, 1241-1245 (1995); Torrens, et al., Neuropeptides, 31, 243-251 (1997)). The subsequent increase in [Ca²⁺]ᵢ promotes the binding of Ca²⁺ to calmodulin (CaM), the resultant Ca²⁺-CaM complex activates MLCK to phosphorylate the MLC of myosin and detrusor smooth muscle contraction occurs (for review see Horowitz, et al., Physiological Reviews, 76(4), 967-1003 (1996). In addition, Y-27632 had no effect on KCl-evoked contractile responses of the isolated male rat urinary bladder. Contractions of the urinary bladder evoked by KCl have previously been shown to be dependent on extracellular Ca²⁺ and are inhibited by Ca² channel antagonists, indicating an involvement of Ca²⁺ influx via voltage sensitive Ca²⁺ channels (Lowe, et al., European Journal of Pharmacology, 195, 273-279 (1991); Visser, et al., Urology Research, 28, 260-268 (2000)). Thus, the mechanisms by which [Ca 2+]ᵢ is increased following exposure to KCl in the urinary bladder are independent of G protein coupled pathways, and the lack of effect of Y-27632 on these responses provides further evidence that ROCK-mediated Ca²⁺ sensitization mechanisms are specifically involved in G protein-coupled contractile responses of this tissue.

ROCK is a serine/threonine protein kinase (Matsui et al., 1996) with two isoforms, namely ROCK I (p160^{ROCK}/ROμ; Ishizaki, et al., EMBO Journal, 15, 1885-1893 (1996)) and ROCK II (ROKμ; Leung, et al., Journal of Biological Chemistry, 270, 29051-29054 (1995)). ROCK I has 64% sequence identity with ROCK II throughout their structures, and the two isoforms share 90% identity in the kinase domain (for review see Amano, et al., Experimental Cell Research, 261, 44-51 (2000). The present study has identified the presence of both ROCK I and ROCK II in the male rat urinary bladder. Furthermore, the levels of both these isoenzymes were higher than those detected in the male rat brain, liver, kidney and skeletal muscle, whilst the levels of ROCK I were higher in the rat aorta than the bladder. Interestingly, the levels of ROCK II in the male rat bladder were higher than those detected in rat aorta. This is the first demonstration of the expression of ROCK in the urinary bladder of the rat, or other species, and the identification of ROCK I and ROCK II in the male rat bladder indicates a role for these isoenzymes in this tissue. The invention provides for such expression patterns in mammals, particularly humans. Furthermore, the observation that Y-27632 (and another ROCK inhibitor HA-1077, see elswhere herein) decreased bladder tone in the absence of contractile responses evoked by agonists provides further evidence for the presence of ROCK in the male rat urinary bladder. The invention further provides that ROCK plays a role in the maintenance of baseline bladder tone in vivo. Y-27632 is a highly selective inhibitor of both isoforms of ROCK with reported Kᵢ values of 0.14 μM (Uehata, et al., Nature, 389, 990-994 (1997)) and 0.22 μ(Ishizaki, et al., Molecular Pharmacology, 57, 976-983 (2000)) for ROCK I and 0.30 μM for ROCK II (Ishizaki, et al., Molecular Pharmacology, 57, 976-983 (2000)). Furthermore, Y-27632 is approximately 200-300 (Uehata, et al., Nature, 389, 990-994 (1997); μ (Ishizaki, et al., Molecular Pharmacology, 57, 976-983 (2000)) and 1250 (Uehata, et al., Nature, 389, 990-994 (1997) fold more selective for ROCK isoforms than protein kinase C (PKC) and MLCK, respectively, which also possess roles in smooth muscle contractility (for review see Horowitz, et al., Physiological Reviews, 76(4), 967-1003 (1996)). Davies, et al., Biochemical Journal, 351, 95-105 (2000) has also shown that Y-27632 has a minimal effect on a large number of other serine/threonine protein kinases. The present invention also provides that the concentration of Y-27632 that was used in the present study (10 μM) inhibits both ROCK I and ROCK II, whilst having no effect on other kinases relevant to smooth muscle contractility, including MLCK and PKC. Studies performed as part of the basis of the invention showed that this concentration of Y-27632 had no effect on contractile responses of the isolated male rat urinary bladder to the PKC activator, phorbol ester (for review see Nambi, et al., Pharmacology Reviews and Communications, 8, 29-38 (1996)), which were inhibited by the PKC antagonist GF109203X (Toullec, et al., Journal of Biological Chemistry, 266, 15771-15781 (1991); unpublished observations). Therefore, one model, that is not limitative of the present invention, indictaes that attenuation of carbachol- and NKA-evoked bladder contractions following Y-27632 treatment is due to be due to an inhibition of ROCK rather than an effect on other kinases in this tissue. An embodiment of the invention is directed to selective inhibitors of ROCK I and/or ROCK II used to treat disease. The effects of Y-27632 on carbachol-evoked bladder contractions in the present study are in agreement with those of Jezior, et al., Pharmacology, 134, 78-87 (2001)) who have demonstrated that Y-27632, in addition to HA-1077, attenuate bethanechol-evoked contractions of isolated rabbit urinary bladder smooth muscle. Further evidence for the presence of Ca²⁺ sensitization mechanisms in urinary bladder smooth muscle is also provided from the observation that carbachol-evoked contractions are still present following depletion of both extracellular and intracellular Ca²⁺ in the rat (Maggi, et al., General Pharmacology, 19, 73-81 (1988)), rabbit (Kishii, et al., Japanese Journal of Pharmacology, 58, 219-229 (1992)) and Yoshimura, et al., International Journal of Urology, 4, 62-67 (1997)). Y-27632 also attenuates muscarinic acetylcholine receptor-mediated contractile responses in other tissues, including airway (Janssen, et al., Journal of Applied Physiology, 91, 1142-1151 (2001)) and gastrointestinal (Sward et al., 2000) smooth muscles. Furthermore, a role for ROCK in NKA-mediated contractile responses has been demonstrated in the isolated human bronchus (Yamagata, et al, Pulmonary Pharmacology and Therapeutics, 13, 25-29 (2000)) with the use of Y-27632 to pharmacologically manipulate this pathway. However, this invention is based, in part, on the new observation of the disclosed effects of Y-27632 on NKA-evoked tone in the urinary bladder.

In addition to the aforementioned experiments investigating the effects of Y-27632 on KCl-evoked bladder contractile responses, experiments were also performed to examine the effects of Y-27632 on α,β-methylene ATP-evoked bladder contractions. α,β-methylene ATP induces contraction of urinary bladder smooth muscle via P2X, receptors (Suzuki, et al., British Journal of Pharmacology, 112, 117-122 (1994)), which mediate both the depolarization of the membrane causing the influx of Ca²⁺ ions through voltage-dependent Ca²⁺ channels and the influx of Ca²⁺ ions directly through the receptor (Suzuki, et al., British Journal of Pharmacology, 112, 117-122 (1994)). Therefore, in addition to experiments using KCl stimulation, the effects of Y-27632 on α,β-methylene ATP-evoked responses can also be used to confirm that ROCK-mediated Ca²⁺ sensitization mechanisms are specifically coupled to G protein-coupled receptor-mediated stimulation and to control for specificity. Surprisingly, Y-27632 significantly attenuated α,β-methylene ATP-evoked contractions in the male rat urinary bladder. To ensure this was not due to a non-selective effect of Y-27632 on bladder smooth muscle contractile mechanisms, the effects of HA-1077 were also examined on α,β-methylene ATP-evoked contractions and a similar inhibitory effect was observed that was not significantly different. HA-1077 is selective inhibitor of both isoforms of ROCK with a reported Kᵢ value of 0.33 μM for ROCK I (Uehata, et al., Nature, 389, 990-994 (1997)) and an IC₅₀ of 1.9 μM for ROCK II (Davies, et al., Biochemical Journal, 351, 95-105 (2000)). Thus, the concentration of HA-1077 used in the present study (10 μM) will inhibit both isoforms of ROCK on the basis of previously published literature. However, Uehata, et al., Nature, 389, 990-994 (1997) reported a Kᵢ value of 9.3 μM for PKC from rat brain, although conversely, Davies, et al., Biochemical Journal, 351, 95-105 (2000) found that 20 μM HA-1077 had no significant inhibitory effects on the human form of this kinase. Furthermore, studies showed that HA-1077 had no inhibitory effects on contractions of the male rat isolated urinary bladder to the PKC activator, phorbol ester. Thus, these results indicate that the effects of Y-27632 and HA-1077 on α,β-methylene ATP-evoked bladder contractions are most likely mediated by a selective effect on ROCK-mediated contractile pathways and are not the result of modulation of other contractile mechanisms in this tissue. Indeed, the lack of effect of Y-27632 on KCl-evoked bladder contractions is consistent with this hypothesis. (The possible effects of Y-27632 and HA-1077 on other kinases are discussed later). However, despite these surprising results, the effects of Y-27632 do exhibit selectivity in the degree of attenuation of G protein-coupled receptor contractile responses (carbachol and NKA) when compared with α,β-methylene ATP-evoked contractile responses (cf. approximately 50% vs 30%) in the male rat urinary bladder. Furthermore, selectivity is also observed between the effects of Y-27632 on G protein-coupled receptor- and KCl-evoked bladder contractile responses. The mechanism by which inhibition of ROCK modulates α,β-methylene ATP-evoked contractions in the isolated male rat urinary bladder remains undetermined. However, one possibility is that Y-27632 affects prostaglandin (PG)-mediated contractile mechanisms. Indeed, endogenous PGs have been reported to modulate the intensity of contraction by stimulants in a number of smooth muscle preparations (Orehek, et al., Journal of Pharmacology and Experimental Therapeutics, 194, 554-564 (1975); Maggi, et al., Journal of Pharmacology and Experimental Therapeutics, 230, 500-513 (1984)) and ATP is a known stimulator of PG synthesis (Brown, et al., European Journal of Pharmacology, 69, 81-86 (1981)). In addition, increases in PG levels have been detected following exposure to ATP in the isolated rabbit urinary bladder (G. F. Anderson, Journal of Pharmacology and Experimental Therapeutics, 220, 347-352 (1982)) and contractile bladder responses to ATP in the rabbit (G. F. Anderson, Journal of Pharmacology and Experimental Therapeutics, 220, 347-352 (1982); Andersson, et al., British Journal of Pharmacology, 70, 443-452 (1980)) and to α,β-methylene ATP in the rat (Naramatsu, et al., British Journal of Pharmacology, 122, 558-562 (1997)) are indomethacin-sensitive. Arachidonic acid (AA), the precursor of PG synthesis, directly activates ROCK in smooth muscle fibres from rabbit femoral artery (Araki, et al., European Journal of Physiology, 441, 596-603 (2001)) and AA-evoked contractile responses of the isolated rabbit pulmonary artery are attenuated by Y-27632 (Fu, et al., FEBS Letters, 440, 183-187 (1998)). In addition, HA-1077 inhibits PG-evoked contractions of the isolated rabbit aorta (Seto, et al., European Journal of Pharmacology, 195, 267-272 (1991)). Therefore, the attenuation of α,β-methylene ATP-evoked responses of the isolated male rat urinary bladder by Y-27632 and HA-1077, may be mediated by a modulatory effect on PG-mediated mechanisms. In this respect, contractions evoked by carbachol and a NK₂ receptor agonist in the isolated rat (Jeremy, et al., Naunyn Schmiedebergs Archives of Pharmacology, 334, 463-467 (1986)) and hamster (Tramontana, et al., Naunyn Schmiedebergs Archives of Pharmacology, 361, 452-459 (2000)) urinary bladder, respectively, have been shown to stimulate PG synthesis. This indicates that the attenuating effects of Y-27632 on responses to these agonists in the urinary bladder may also involve effects on PG-mediated pathways. Alternatively, other Y-27632 and HA-1077-sensitive enzymes may play a role in α,β-methylene ATP-evoked contractions of the male rat urinary bladder. However, protein kinase C-related protein kinase 2 (PRK2; Mukai, et al., Biochemical and Biophysical Research Communications, 199, 897-904 (1994)), a Rho effector with roles in the regulation of the cell cytoskeleton (Maesaki, et al., Molecular Cell, 4, 793-803 (1999); Vincent, et al., Molecular Cell Biology, 17, 2247-2256 (1997)), is the only other known kinase other than ROCK that has been shown to be inhibited by both Y-27632 and HA-1077 (Davies, et al., Biochemical Journal, 351, 95-105 (2000)). The concentration (10 μ) of the inhibitors used in the present study will inhibit PRK2 on the basis of the aforementioned previously published literature. Y-27632 has also been demonstrated to inhibit PRK1 (also termed protein kinase N), another member of the PRK superfamily, with a Kᵢ value of 3.1 μM, although no data on the effects of HA-1077 on this kinase has been published. However, the presence and possible role of these kinases in the bladder remain to be determined. An effect of Y-27632 and HA-1077 on further kinases and enzymes present in smooth muscle to attenuate α,β-methylene ATP-evoked responses cannot be ruled out. Furthermore, one model holds that these inhibitors act on other mechanisms involved in α,β-methylene ATP-evoked bladder contractions, for example a direct inhibitory effect on the P2X₁ receptor, but this model in no way limits the scope of the present invention.

The contractile response to electrical field stimulation in the isolated rat and guinea-pig urinary bladder are inhibited by a combination of atropine and desensitization of P2X receptors with α,β-methylene ATP, indicating that acetylcholine and ATP are co-released from the nerve terminals to initiate contraction, mediated by muscarinic and P2X receptors, respectively (Brading & Williams, 1990). It has been proposed that each of these components differs in the nature of their effects on the bladder during micturition, with ATP being largely responsible for the initiation of voiding, while cholinergic transmission affects the maintenance of voiding (Theobald, 1995). Indeed, the contractile response to a single nerve stimulus in the rat detrusor is biphasic, with the second phase being preferentially blocked by atropine (Maggi, et al., Journal of Autonomic Pharmacology, 5, 221-230 (1985); Bhat, et al., British Journal of Pharmacology, 96, 837-842 (1989); Brading, et al., British Journal of Pharmacology, 99, 493-498 (1990)). Thus, following preliminary studies of the inventors demonstrating the presence of a degree of selectivity in the effects of Y-27632 between cholinergic (carbachol) and purinergic (α,β-methylene ATP) bladder contractile responses, experiments were performed to investigate if this selectivity could also be demonstrated in contractile responses of the isolated male rat bladder to electrical stimulation. Electrical stimulation evoked contractile responses of the male rat urinary bladder that were biphasic in nature, comprising a rapid increase in bladder tension (measured as the amplitude of contraction), followed by a secondary sustained contraction (measured as the AUC). The secondary contractile phase was preferentially attenuated by atropine in comparison to desensitization of P2X receptors with α,β-methylene ATP, consistent with the results of previous studies in this species (Maggi, et al., Journal of Autonomic Pharmacology, 5, 221-230 (1985); Brading, et al., British Journal of Pharmacology, 99, 493-498 (1990)). Furthermore, Y-27632 significantly attenuated the secondary sustained contractile component to a similar degree to atropine and to a greater extent than α,β-methylene ATP. The concomitant treatment of Y-27632 with atropine also inhibited the secondary phase of the contractions to a greater degree than a combination of Y-27632 with α,β-methylene ATP. This is consistent with the view that the contractile effects of cholinergic stimulation in the bladder, represented by a sustained contractile response to electrical stimulation in vitro, involve ROCK-mediated pathways. The amplitude of contractile responses to electrical field stimulation were attenuated to a similar degree by both atropine and α,β-methylene ATP, indicating an involvement of both cholinergic and purinergic transmission. However, Y-27632 similarly attenuated this initial rapid bladder contractile response to electrical stimulation, and further, to a significantly greater degree than the effects of atropine alone, but to a similar degree to the effects of α,β-methylene ATP. Thus, these results provide further evidence that ROCK plays a role in P2X receptor-mediated contractile responses of the isolated male rat urinary bladder. Furthermore, the present study shows that selectivity, at least in part, can also be demonstrated in the effects of Y-27632 on the biphasic contractile responses to electrical stimulation in the male rat urinary bladder.

The results of the present study are particularly surprising with regard to the pharmacological treatment of lower urinary tract dysfunction or overactive bladder. Current therapy of various forms of bladder disorders, including overactive bladder (for review see Wyndaele, et al., British Journal of Urology, 88, 135-140 (2001)), is primarily directed to the use of anti-muscarinics, which are often limited by their adverse effects, including dry mouth and constipation (for review see Chapple, et al., Urology, 55, 33-46 (2000)). Thus, ROCK inhibitors are potentially useful in the treatment of bladder dysfunction, working downstream of the muscarinic receptor, in that they preferred compounds lack these side effects, among other improved qualities. However, a number of studies have indicated that although ATP may possess a limited role in human bladder smooth muscle contractility under normal physiological conditions, purinergic transmission may have significant implications in disease states in this tissue (Saito, et al., British Journal of Urology, 72, 298-302 (1993); Bayliss, et al., Journal of Urology, 162, 1833-1839 (1999)). Thus, ROCK inhibitors may also exert therapeutic effects on the pathological changes associated with bladder dysfunction, via modulation of P2X-mediated contractile mechanisms.

The aforementioned study identified the presence of ROCK I and ROCK II in the male rat urinary bladder, and has demonstrated that ROCK-induced Ca²⁺ sensitization plays a role in G protein coupled responses in this tissue. Furthermore, a role for ROCK-mediated pathways in P2X-mediated contractile responses of this tissue has also been indicated, although the exact mechanism(s) remain to be determined. An embodiment of the invention provides a dual role of ROCK-mediated contractile mechanisms in both cholinergic and purinergic transmission in the urinary bladder and indicates that ROCK inhibitors, including those identified by ROCK screening as provided herein, are therapeutically useful in the treatment of lower urinary tract disorders and/or overactive bladder associated with changes in the pharmacology of bladder smooth muscle contractility in mammals, particularly in humans.

An further embodiment of ROCK as used in the methods of the invention relates to polypeptides including: (a) an isolated polypeptide encoded by a polynucleotide comprising the sequence of ROCK;
(b) an isolated polypeptide comprising a polypeptide sequence having at least 95%, 96%, 97%, 98%, or 99% identity to the polypeptide sequence of ROCK; (c) an isolated polypeptide comprising the polypeptide sequence of ROCK; (d) an isolated polypeptide having at least 95%, 96%, 97%, 98%, or 99% identity to the polypeptide sequence of ROCK; (e) the polypeptide sequence of ROCK; and
(f) an isolated polypeptide having or comprising a polypeptide sequence that has an Identity Index of 0.95, 0.96, 0.97, 0.98, or 0.99 compared to the polypeptide sequence of ROCK; (g) fragments and variants of such polypeptides in (a) to (f).

Polypeptides useful in the methods of the present invention are members of the ROCK family of polypeptides. They are therefore of interest because they have been linked to smooth muscle contractility, and diseases related thereto, as further set forth elsewhere herein.

The biological properties of ROCK are herein referred to as “biological activity of ROCK” or “ROCK activity,” certain of which are described in detail herein, others being known in the art. Preferably, a polypeptide useful in the methods of the present invention exhibits at least one biological activity of ROCK.

Other polypeptides useful in the methods of the present invention also include variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Preferred fragments of polypeptides useful in the methods of the present invention include an isolated polypeptide comprising an amino acid sequence having at least 30, 50 or 100 contiguous amino acids from the amino acid sequence of ROCK, or an isolated polypeptide comprising an amino acid sequence having at least 30, 50 or 100 contiguous amino acids truncated or deleted from the amino acid sequence of ROCK. Preferred fragments are biologically active fragments that mediate the biological activity of ROCK, including those with a similar activity or an improved activity, or with a decreased undesirable activity. Also preferred are those fragments that are antigenic or immunogenic in an animal, especially in a human.

Fragments of polypeptides useful in the methods of the invention may be employed for producing the corresponding full-length polypeptide by peptide synthesis; therefore, these variants may be employed as intermediates for producing the full-length polypeptides of the invention. The polypeptides of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Further polypeptides useful in the methods of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

In a further aspect, the present invention relates to methods comprising ROCK polynucleotides. Such polynucleotides include: (a) an isolated polynucleotide comprising a polynucleotide sequence having at least 95%, 96%, 97%, 98%, or 99% identity to the polynucleotide sequence of ROCK;
(b) an isolated polynucleotide comprising the polynucleotide of ROCK;
(c) an isolated polynucleotide having at least 95%, 96%, 97%, 98%, or 99% identity to the polynucleotide of ROCK; (d) the isolated polynucleotide of ROCK; (e) an isolated polynucleotide comprising a polynucleotide sequence encoding a polypeptide sequence having at least 95%, 96%, 97%, 98%, or 99% identity to the polypeptide sequence of ROCK; (f) an isolated polynucleotide comprising a polynucleotide sequence encoding the polypeptide of ROCK; (g) an isolated polynucleotide having a polynucleotide sequence encoding a polypeptide sequence having at least 95%, 96%, 97%, 98%, or 99% identity to the polypeptide sequence of ROCK; (h) an isolated polynucleotide encoding the polypeptide of ROCK; (i) an isolated polynucleotide having or comprising a polynucleotide sequence that has an Identity Index of 0.95, 0.96, 0.97, 0.98, or 0.99 compared to the polynucleotide sequence of ROCK; (j) an isolated polynucleotide having or comprising a polynucleotide sequence encoding a polypeptide sequence that has an Identity Index of 0.95, 0.96, 0.97, 0.98, or 0.99 compared to the polypeptide sequence of ROCK; and polynucleotides that are fragments and variants of the above mentioned polynucleotides or that are complementary to above mentioned polynucleotides, over the entire length thereof.

Preferred fragments of polynucleotides useful in the methods of the present invention include an isolated polynucleotide comprising an nucleotide sequence having at least 15, 30, 50 or 100 contiguous nucleotides from the sequence of a ROCK polynucleotide, or an isolated polynucleotide comprising an sequence having at least 30, 50 or 100 contiguous nucleotides truncated or deleted from the sequence of a ROCK polynucleotide.

Preferred variants of polynucleotides useful in the methods of the present invention include splice variants, allelic variants, and polymorphisms, including polynucleotides having one or more single nucleotide polymorphisms (SNPs).

Polynucleotides useful in the methods of the present invention also include polynucleotides encoding polypeptide variants that comprise the amino acid sequence of ROCK and in which several, for instance from 50 to 30, from 30 to 20, from 20 to 10, from 10 to 5, from 5 to 3, from 3 to 2, from 2 to 1 or 1 amino acid residues are substituted, deleted or added, in any combination.

In a further aspect, the present invention provides polynucleotides useful in the methods herein that are RNA transcripts of the DNA sequences of the present invention. Accordingly, there is provided an RNA polynucleotide that: (a) comprises an RNA transcript of the DNA sequence encoding the polypeptide of ROCK; (b) is the RNA transcript of the DNA sequence encoding the polypeptide of ROCK; (c) comprises an RNA transcript of the DNA sequence of ROCK; or (d) is the RNA transcript of the DNA sequence of ROCK; and RNA polynucleotides that are complementary thereto.

Preferred polypeptides and polynucleotides useful in the methods of the present invention are expected to have, inter alia, similar biological functions/properties to their homologous polypeptides and polynucleotides. Furthermore, preferred polypeptides and polynucleotides of the present invention have at least one ROCK activity.

Further polynucleotides useful in the methods present invention may be obtained using standard cloning and screening techniques from a cDNA library derived from mRNA in cells of human bladder smooth muscle (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides useful in the methods of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al, Proc Natl Acad Sci USA (1989) 86: 821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Polynucleotides useful in the methods that are identical, or have sufficient identity to a polynucleotide sequence of ROCK, may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from species other than human or rat) that have a high sequence similarity to ROCK, typically at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide useful in the methods of the present invention, including homologs from species other than human or rat, may be obtained by a process comprising the steps of screening a library under stringent hybridization conditions with a labeled probe having the sequence of a ROCK polynucleotide or a fragment thereof, preferably of at least 15 nucleotides; and isolating full-length cDNA and genomic clones containing said polynucleotide sequence. Such hybridization techniques are well known to the skilled artisan. Preferred stringent hybridization conditions include overnight incubation at 420C in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence of ROCK or a fragment thereof, preferably of at least 15 nucleotides.

Recombinant polypeptides useful in thye methods of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al. (ibid). Preferred methods of introducing polynucleotides into host cells include, for instance, calcium phosphate transfection, DEAE-dextran mediated transfection, transvection, micro-injection, cationic lipid-mediated transfection, electroporation, transduction, scrape loading, ballistic introduction or infection.

Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express the polynucleotide to produce a polypeptide in a host may be used. The appropriate polynucleotide sequence may be inserted into an expression system by any of a variety of well-known and routine techniques, such as, for example, those set forth in Sambrook et al., (ibid). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides useful in the methods of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides useful in the methods of the present invention may be used as diagnostic reagents, through detecting mutations in the associated gene. Detection of a mutated form of the gene characterized by the polynucleotide of ROCK in the cDNA or genomic sequence and which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled ROCK nucleotide sequences. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985) 230: 1242). Sequence changes at specific locations may also be revealed by nuclease protection assays, such as RNase and S1 protection or the chemical cleavage method (see Cotton et al., Proc Natl Acad Sci USA (1985) 85: 4397-4401).

An array of oligonucleotide probes comprising ROCK polynucleotide sequence or fragments thereof can be constructed to conduct efficient screening of e.g., genetic mutations and variants. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention (refer also to the Examples). Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radio-immunoassays, competitive-binding assays, Western Blot analysis and ELISA assays.

Thus in another aspect, the present invention relates to a diagnostic kit comprising: (a) a polynucleotide of the present invention, preferably the nucleotide sequence of ROCK, or a fragment or an RNA transcript thereof; (b) a nucleotide sequence complementary to that of (a); (c) a polypeptide of the present invention, preferably the polypeptide of ROCK or a fragment thereof; or (d) an antibody to a polypeptide of the present invention, preferably to the polypeptide of ROCK.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are also valuable tools for tissue expression studies. Such studies allow the determination of expression patterns of polynucleotides of the present invention which may give an indication as to the expression patterns of the encoded polypeptides in tissues, by detecting the mRNAs that encode them. The techniques used are well known in the art and include in situ hybridization techniques to clones arrayed on a grid, such as cDNA microarray hybridization (Schena et al, Science, 270, 467-470, 1995 and Shalon et al, Genome Res, 6,639-645, 1996) and nucleotide amplification techniques such as PCR. A preferred method uses the TAQMAN (Trade mark) technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

ROCK I polypeptides of the present invention are expressed in bladder and aorta (refer to Example 1). ROCK 2 polypeptides of the present invention are expressed in bladder (refer to Example 1).

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256: 495-497), the trioma technique, the human B-cell hybridoma technique (Kozbor et al., Immunology Today (1983) 4: 72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down in the stomach, it is preferably administered parenterally (for instance, subcutaneous, intra-muscular, intravenous, or intra-dermal injection). Formulations suitable for parenteral administration include aqueous and non-aqueous sterile injection solutions that may contain anti-oxidants, buffers, bacteriostats and solutes that render the formulation isotonic with the blood of the recipient; and aqueous and non-aqueous sterile suspensions that may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2): Chapter 5 (1991)) or a small molecule. Such small molecules preferably have a molecular weight below 2,000 daltons, more preferably between 300 and 1,000 daltons, and most preferably between 400 and 700 daltons. It is preferred that these small molecules are organic molecules.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labeled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring a ROCK activity in the mixture, and comparing the ROCK activity of the mixture to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

Fusion proteins, such as those made from Fc portion and ROCK polypeptide, as hereinbefore described, can also be used for high-throughput screening assays to identify antagonists for the polypeptide of the present invention (see D. Bennett et al., J Mol Recognition, 8: 52-58 (1995); and K. Johanson et al., J Biol Chem, 270(16): 9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology and ROCK gene. The art of constructing transgenic animals is well established. For example, the ROCK gene may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention.

Screening kits for use in the above described methods form a further aspect of the present invention. Such screening kits comprise: (a) a polypeptide of the present invention; (b) a recombinant cell expressing a polypeptide of the present invention; (c) a cell membrane expressing a polypeptide of the present invention; or (d) an antibody to a polypeptide of the present invention; which polypeptide is preferably that of ROCK.

It will be appreciated that in any such kit, (a), (b), (c) or (d) may comprise a substantial component.

### Glossary

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence of ROCK.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”- For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Needleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25: 389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448,1988, available as part of the Wisconsin Sequence Analysis Package).

Preferably, the BLOSUM62 amino acid substitution matrix (Henikoff S and Henikoff J G, Proc. Nat. Acad. Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:
nₐ≦xₐ−(xₐ·I),
in which:
nₐ is the number of nucleotide or amino acid differences,
xₐ is the total number of nucleotides or amino acids in ROCK or ROCK, respectively,
I is the Identity Index,
· is the symbol for the multiplication operator, and
   in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotideor polypeptide that within the same species which is functionally similar.

“Fusion protein” refers to a protein encoded by two, often unrelated, fused genes or fragments thereof. In one example, EP-A-0 464 533-A discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

“NKA” means neurokinin A.

“ROCK” herein refers to a rho kinase, including, as known in the art, ROCK 1 and/or ROCK 2 genes or proteins, particularly mammalian ROCK, especially human ROCK. For example, human ROCK is described in U.S. Pat. No. 5,906,819 (human and bovine ROCK).

“Modulates” means in reference to an activity herein, resulting in a change in an amount, and/or quality, and/or effect of a particular response and/or activity. Both increases and/or decreases in a response and/or activity are included.

### EXAMPLES

The invention is further illustrated by way of the following examples which are intended to elucidate the invention. These examples are not intended, nor are they to be construed, as limiting the scope of the invention. It will be clear that the invention may be practised otherwise that as particularly described herein. Numerous modifications and variations of the present invention are possible in view of the teachings herein and, therefore, are within the scope of the invention. The examples below are carried out using standard techniques, which are well known and routine to those of skill in the art, except where otherwise described in detail.

### Example 1

### Expression Studies

Five male Sprague-Dawley rats (350-600 g; Charles River, U.S.A.) were anaesthetized with isoflurane (4% in 100% oxygen) and killed by cervical dislocation. The following tissues were exposed and removed; urinary bladder, aorta, heart, liver, kidney, brain and skeletal muscle (diaphragm). Isolation of total RNA from rat tissues was performed as described previously (Sambrook et al., 1989). Real-time quantitative polymerase chain reaction (PCR) analysis (Heid et al., 1986) was used to determine the relative levels of ROCK I and ROCK II mRNA in rat tissues. 1-2 μg of total RNA of each sample was treated with RQ Dnase I (Promega Biotech., Madison, U.S.A.) prior to reverse transcription. Each reverse transcripted cDNA was diluted to 100 μl and 5 μl was loaded to each PCR reaction. Reverse transcription and PCR reactions were performed according to the manufacturers instructions (Applied Biosystems, Foster City, U.S.A.). ROCK I or ROCK II sequence-specific amplification was detected with an increasing fluorescent signal of FAM reporter dye during the amplification cycle. A rodent glyceraldehyde-3-phosphate dehydrogenase (GAPDH) primer and probe set obtained from Applied Biosystems was used to amplify the mRNA level of GAPDH. This amplification was included in the same reaction on all samples tested as an internal control of variations in RNA amounts. Each sequence specific amplification was quadruplicated. Levels of the different mRNAs were subsequently normalized to GAPDH mRNA levels, and the relative mRNA level of each tissue was then compared to that of heart tissue (whilst arbitrarily setting the mRNA heart level as 1.0). Oligonucleotide primers and Taqman probes were designed using Primer Express software (Applied Biosystems) and were synthesized by Applied Biosystems. Sequences of forward primers, reverse primers, and probes were:

ROCK I—

| | |
|---|---|
| forward primer- | AGGCCTGTGCCAAACCTTT |
| reverse primer- | TGGTCCCTGTGGGACTTAACA |
| Taqman probe- | CCGCCTGCCCTAGAGTGTCGAAGA |

ROCK II—

| | |
|---|---|
| forward primer- | CCCGATCATCCCCTAGAACC |
| reverse primer- | TTGGAGCAAGCTGTCGACTG |
| Taqman probe- | CAACAAAACCAGTCCATTCGGCGGC |

A. Results of Expression Studies

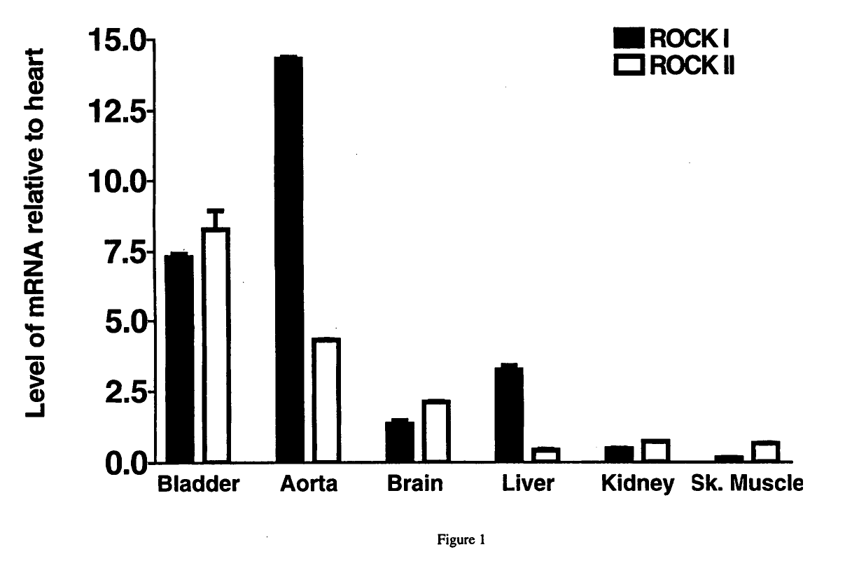

High levels of both ROCK I and ROCK II mRNA were detected in male rat urinary bladder (FIG. 1). In comparison with the levels of ROCK I and ROCK II in other rat tissues, ROCK I mRNA levels were highest in the aorta, followed by a high level of expression in the bladder. Conversely, ROCK II levels in the male rat bladder were approximately twice as high as those detected in the male rat aorta. The levels of ROCK I and ROCK II in the male rat brain, liver, kidney and skeletal muscle were low in comparison to the levels of these kinases in the male rat bladder and aorta.

### Example 2

### Organ Bath Studies

A. General Preparation

Sixty-seven male Sprague-Dawley rats (350-600 g; Charles River, U.S.A.) were anaesthetized with isoflurane (4% in 100% oxygen) and killed by cervical dislocation. Through a midline incision the bladder was exposed, removed, and cut longitudinally to form two roughly equally sized bladder strips. Each bladder strip was then mounted longitudinally under Ig resting tension in 15 ml tissue baths. The tissues were bathed with Krebs solution of the following composition (in mM): NaCl 118; KCl 4.7; NAHCO₃ 25; KH₂PO_{4 1.2}; MgSO₄ 0.58; CaCl₂ 2.5 and glucose 11. The temperature of the baths were maintained at 37° C. using a heater circulator system (VWR1130; VWR Scientific Products, U.S.A.) and were aerated with 95% O₂ and 5% CO₂. Tissues were stimulated using an electrical stimulator (S88; Astro-Med Inc., U.S.A.).

B. Experimental Protocols

All tissues were equilibrated under 1 g resting tension for a period of 60 min, during which the bathing medium was changed several times. For experiments using carbachol and neurokinin A (NKA) as agonists, tissues were exposed to 100 mM potassium chloride (KCl) and allowed to reach peak contractions, to which responses to carbachol and NKA could be normalized. The tissues were then washed over 60 min, and then incubated with Y-27632 or vehicle for a period of 45 min. Concentration-response curves to carbachol or NKA were then constructed. In experiments using KCl as the contractile test substance, tissues were incubated with Y-27632 or vehicle for 45 min following the 60 min equilibration period and concentration-response curves to KCl were then constructed. In experiments investigating the effects of Y-27632 on (α,β-methylene ATP-evoked responses, α,β-methylene ATP was added to the tissues after the equilibration period, allowed to reach peak contractile responses and then washed over 60 min (to avoid desensitization of responses). Tissues were then incubated with antagonist or vehicle for 45 min and second challenges to α,β-methylene ATP were performed. For electrical stimulation experiments, control frequency-response curves were carried out using the following parameters: 150 volts, 0.5 ms pulse width for 30 s, at 15 min intervals, at frequencies of 2, 4, 8 and 16 Hz. Tissues were washed between each frequency of stimulation. Tissues were then incubated with antagonist or vehicle for 45 min and second frequency-response curves were constructed. At the end of all experiments, rat urinary bladder strips were incubated with tetrodotoxin (TTX; 1 μM) for 15 min and third frequency-response curves were performed to confirm that responses to electrical stimulation in these tissues were the result of action potential generation. In experiments using α,β-methylene ATP as a desensitizing agonist of P2X receptors, following construction of control frequency-response curves, α,β-methylene ATP was added to the bath and incubated for 10 min. Second challenges to α,β-methylene ATP were then performed to confirm the desensitization of contractile responses to this agonist, following which further antagonists were added and incubated for 45 min. Second frequency-response curves were then performed as described above.

C. Data Capture and Analysis

Changes in bladder tensions were measured by means of 50 g isometric force transducers (TSD125C; Biopac Systems Inc., U.S.A.), acquired (20 samples per second) by a MP 100 WSW interface (Biopac Systems Inc., U.S.A.) and analyzed off-line using AcqKnowledge version 3.5.7 software (Biopac Systems Inc., U.S.A.). The amplitude (g) of bladder contractions evoked by application of agonists were measured. For concentration-response curves this measurement was made after peak responses to each concentration of agonist had been reached. Changes in bladder tensions evoked by agonists were expressed as percentages of the contractile response to 100 mM KCl in each tissue (where the contractile test substance used was KCl, 100 mM was also the maximal response and concentration used in concentration-response curves) and were compared with vehicle controls by unpaired Student's t-test. The amplitude (g) and area under the curve (AUC) of the contractile bladder responses to electrical field stimulation were measured. Baseline bladder tensions were measured 2 min before administration of test substances, and changes in resting bladder tensions from baseline caused by test substances were measured at 15 min intervals over the 45 min incubation period. Changes in baseline bladder tensions and bladder contractile responses to electrical stimulation were expressed as percentage changes before and after the administration of antagonists, and compared with vehicle controls by unpaired Student's t-test. P values of less than 0.05 were considered indicative of a statistically significant difference. All values are mean±s.e.mean.

D. Drugs and Solutions

Drugs and chemicals were obtained from the following sources: Y-27632 ((+)-(R)-trans-4-(1-amino-ethyl)-N-(4-pyridyl) cyclohexanecarboxamide dihydrochloride monohydrate) and fasudil (hydrochloride; HA-1077) from Tocris Cookson Ltd, U.S.A.; carbamylcholine chloride (carbachol), Neurokinin A, potassium chloride, α,β-methylene ATP, atropine (sulfate) and tetrodotoxin from Sigma Aldrich Chemicals, U.S.A. Krebs ringer solution was made in house (GlaxoSmithKline, U.S.A.). All drugs were dissolved in distilled water. Test substances were added to baths in a maximum of 150 μl volume and the concentrations reported are final bath concentrations.

E. Results of Organ Bath Studies

(1) Effects of 10 μM Y-27632 on Carbachol- and NKA-Evoked Bladder Contractions

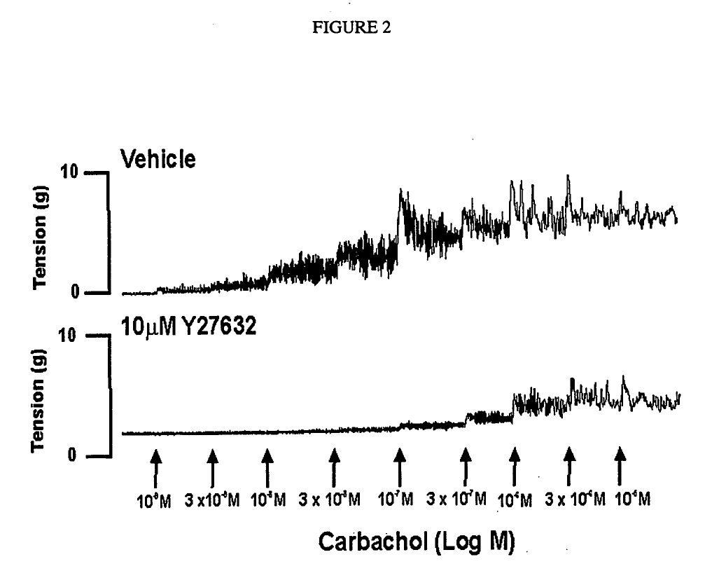

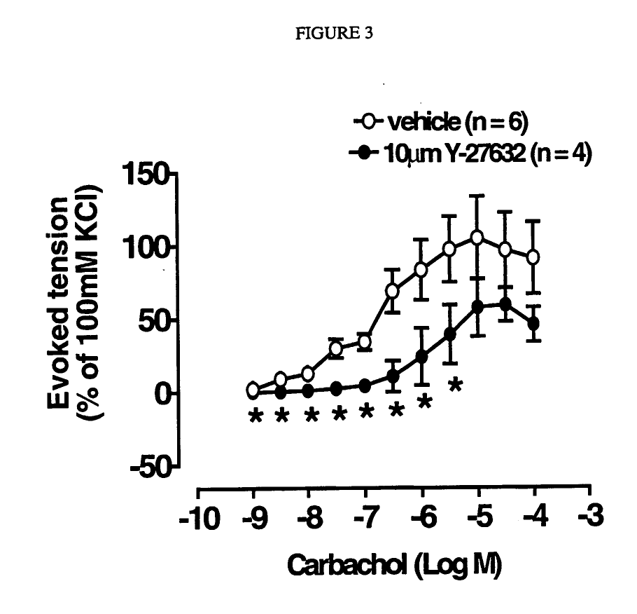

Cumulative additions of carbachol (10⁻⁹-10⁻⁴M) evoked sustained concentration-dependent contractions of isolated male rat urinary bladder strips (FIGS. 2 and 3). The maximal response to carbachol in vehicle experiments was 5.3±0.9 g at 10⁻⁵ M carbachol, which was 105.0±28.2% of the response to 100 mM KCl (FIGS. 2 and 3; n=6). The carbachol EC₅₀ in the absence of antagonist was 183.5±68.9 nM. Preliminary studies demonstrated that pretreatment with 1 and 3 μM Y-27632 had no effect on carbachol-evoked contractions (unpublished observations). However, pretreatment with 10 μM Y-27632 significantly attenuated carbachol-evoked contractions at concentrations between 10⁻⁹-3×10⁻⁶ M carbachol, with an inhibition of 58.1±10.5% at 3×10⁻⁶ M (FIGS. 2 and 3; n=4). At concentrations of 10⁻⁵-10⁻⁴ carbachol, contractions were attenuated by Y-27632, although this inhibition was not significant. The carbachol EC₅₀ in the presence of Y-27632 was 1034.3±336.1 nM, with a calculated agonist dose ratio (DR) of 5.63 and antagonist affinity (pKb) of 5.67. From these preliminary experiments and previously published data on the selectivity of Y-27632 (see discussion), a concentration of 10 μM Y-27632 was used in subsequent experiments.

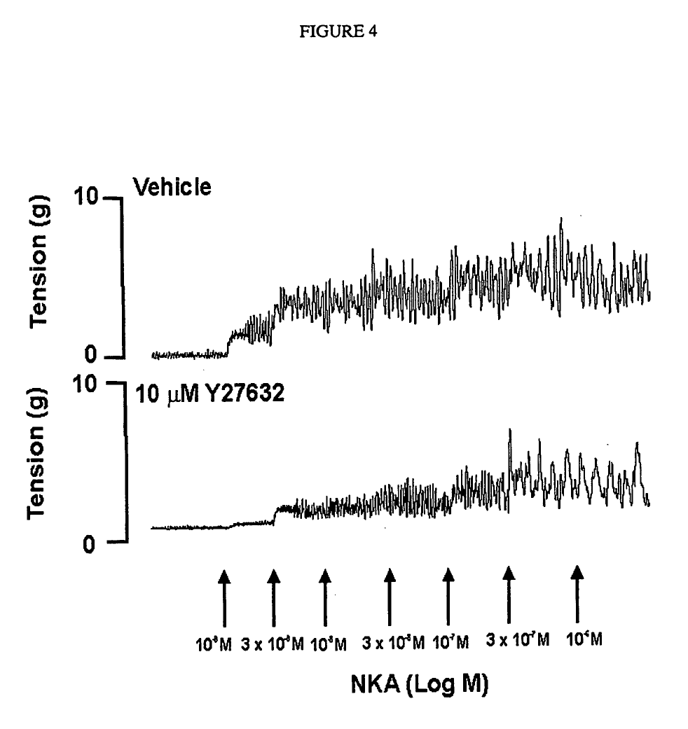

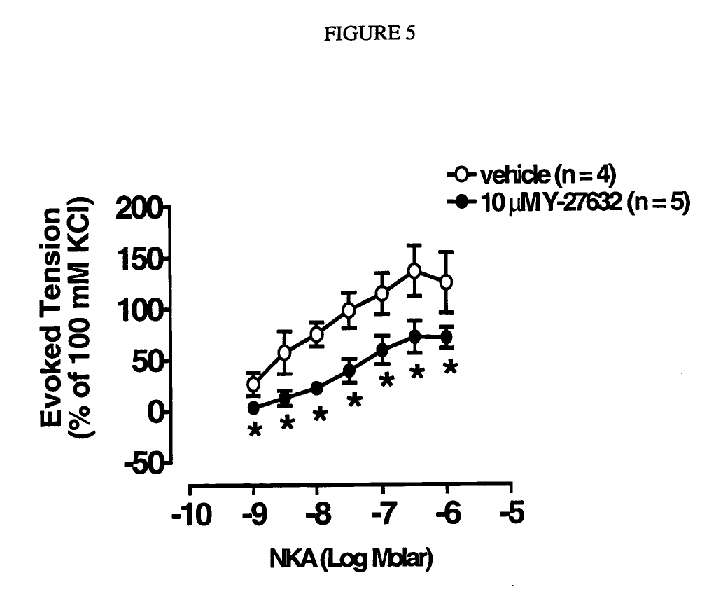

Cumulative additions of NKA (10⁻⁹-10⁻⁶) evoked sustained concentration-dependent contractions of isolated male rat urinary bladder strips (FIGS. 4 and 5). The maximal response to NKA in vehicle experiments was 4.2±0.4 g at 3×10⁻⁷ M NKA, which was 136.2±24.8% of the response to 100 mM KCl (FIGS. 4 and 5; n=4). The NKA EC₅₀ in the absence of antagonist was 6.5±3.9 nM. Pretreatment with 10 μM Y-27632 significantly attenuated NKA-induced contractions at all concentrations of agonist with an inhibition of 68.6±12.7% at 3×10⁻⁷M NKA (FIGS. 4 and 5; n=5). The NKA EC₅₀ in the presence of Y-27632 was 32.4 9.4 nM, with a calculated agonist dose ratio (DR) of 4.98 and antagonist affinity (pKb) of 5.60.

(2) Effects of 10 μM Y27632 on KCl-Evoked Bladder Contractions

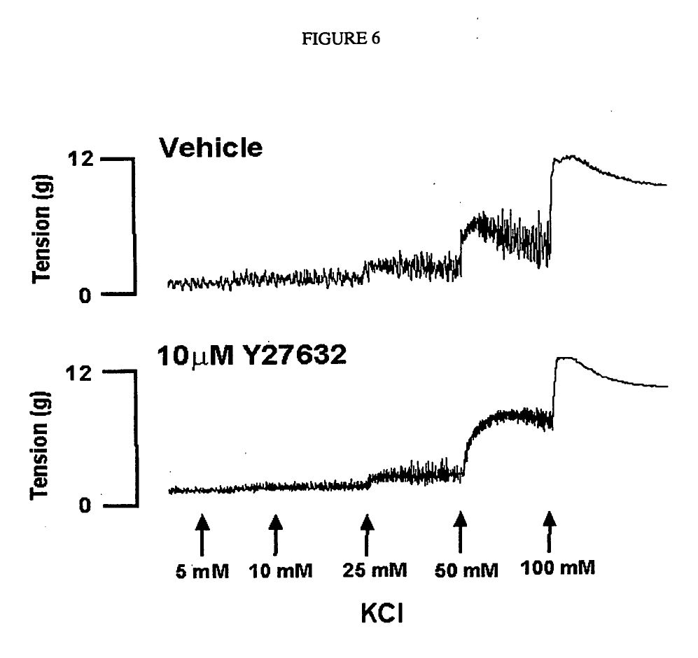

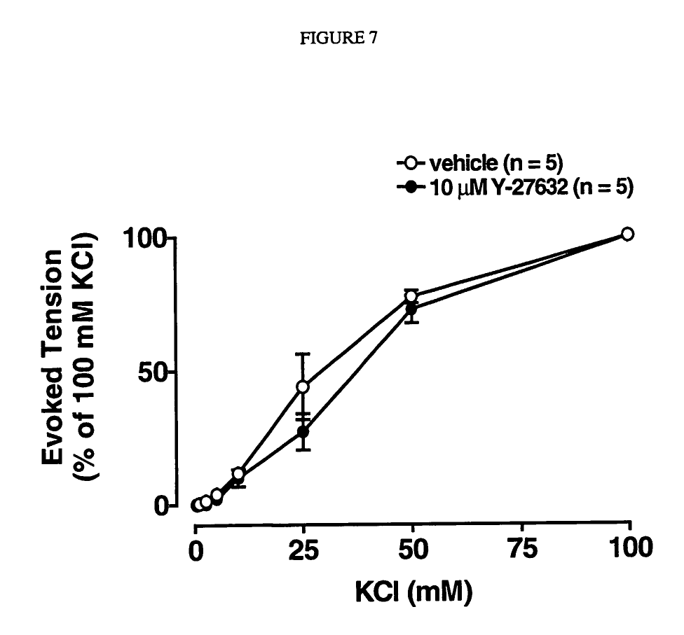

Cumulative additions of KCl (1-100 mM) induced sustained concentration-dependent contractions of isolated male rat urinary bladder strips at concentrations of 10 mM and above (FIGS. 6 and 7). The maximal response to KCl in vehicle experiments was 8.7±0.5 g at 100 mM KCl (n=5). Concentrations of KCl higher than 100 mM evoked relaxations of the isolated male rat urinary bladder (unpublished observations). The KCl EC₅₀ in the absence of antagonist was 31±4.3 mM. Pretreatment with 10 μM Y-27632 had no effect on the contractile response to KCl in any of the bladder strips tested (FIGS. 6 and 7; n=5).

(3) Effects of 10 μM Y-27632 and 10 μM HA-1077 on α,β-Methylene ATP-Evoked Bladder Contractions

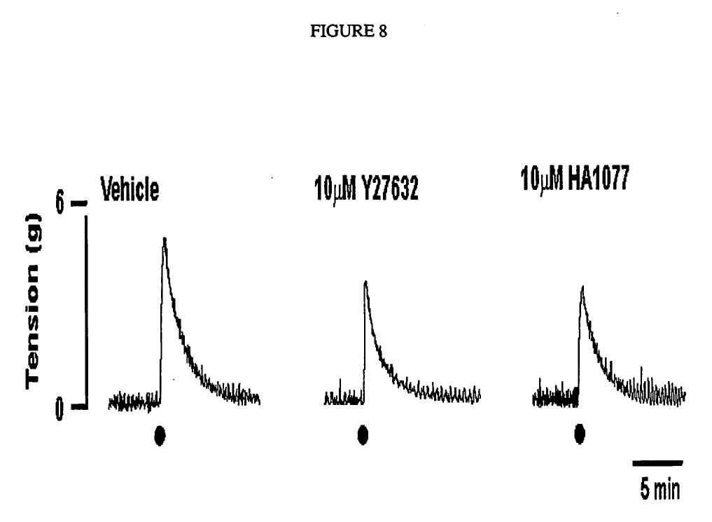

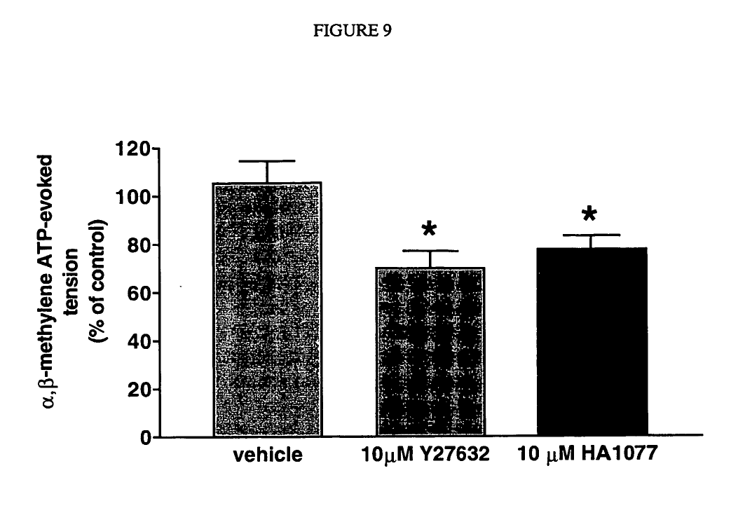

Addition of 10 μM α,β-methylene ATP evoked contractile responses of isolated male rat urinary bladder strips that had a mean amplitude of 3.9±0.5 g (FIG. 8; n=14). Interestingly, Y-27632 (10 μM) significantly attenuated α,β-methylene ATP-evoked bladder contractions by 30.0±7.2% (FIGS. 8 and 9; n=4). To further investigate this unexpected experimental result, the effects of another ROCK inhibitor HA-1077 (fasudil hydrochloride; Davies et al., 2000) were also examined on α,β-methylene ATP-evoked bladder contractions in the isolated male rat urinary bladder. HA-1077 (10 μM) also significantly attenuated α,β-methylene ATP-evoked bladder contractions by 22.1±5.7% (FIGS. 8 and 9; n=5). This inhibition by HA-1077 was not significantly different from the inhibition of α,β-methylene ATP-evoked bladder contractions following treatment with Y-27632.

(4) Effects of 10 μM Y-27632 and Combinations of Y-27632, Atropine and α,β-Methylene ATP on Electrically-Evoked Bladder Contractions

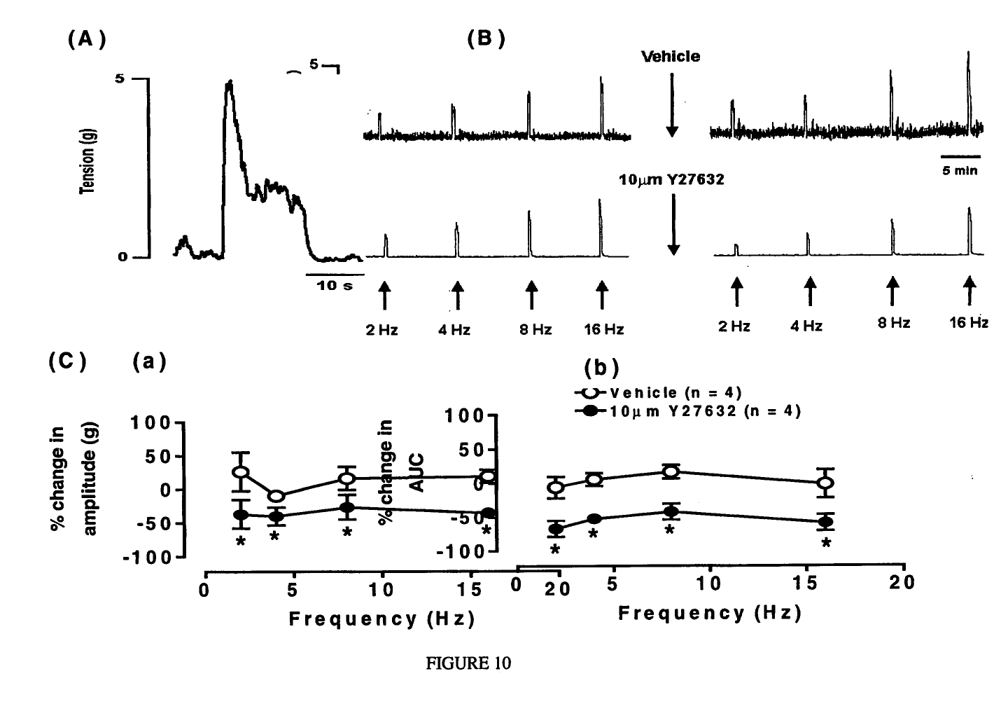

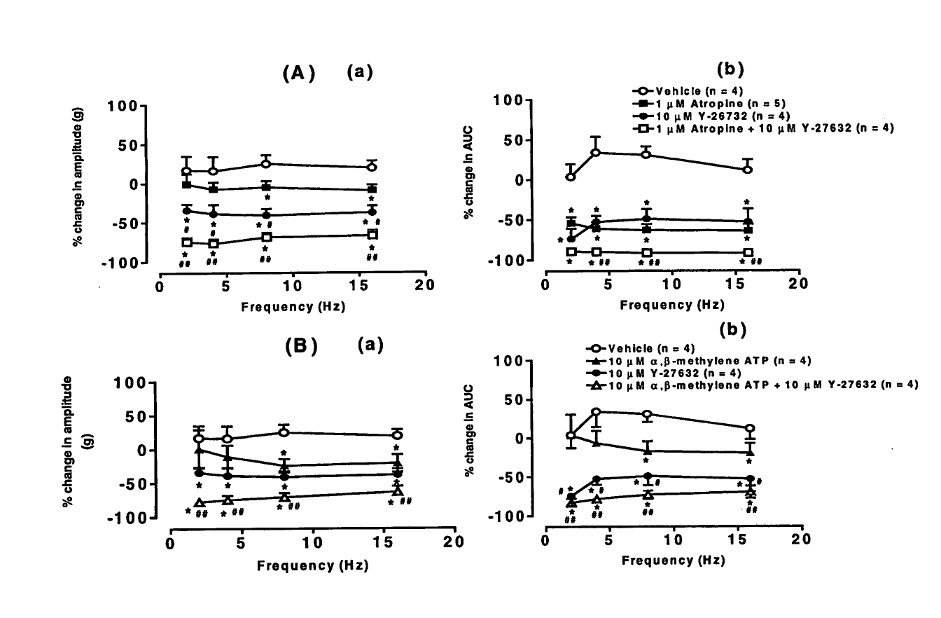

Electrical stimulation evoked frequency-dependent contractions of the isolated male rat urinary bladder at 2, 4, 8 and 16 Hz, that were biphasic, comprising a rapid increase in bladder tension followed by a sustained contraction (FIG. 10A). The mean amplitude of electrically-evoked bladder contractions from control frequency-response curves for all experimental groups were 2.0±0.4 g, 2.9±0.5 g, 3.9±0.7 g and 4.8±0.8 g at 2, 4, 8 and 16 Hz, respectively. Furthermore, the mean AUC for these contractions were 45.9±10.3, 67.7±15.0, 90.3±18.5 and 108.9±21.0 at 2, 4, 8 and 16 Hz, respectively (n=26). Y-27632 (10 μM) significantly attenuated both the amplitude and AUC of electrically-evoked bladder contractions at all frequencies of stimulation (FIGS. 10B & C; n=4). There was no significant difference between the effects of Y-27632 on the amplitude and AUC of electrically-evoked bladder contractions.

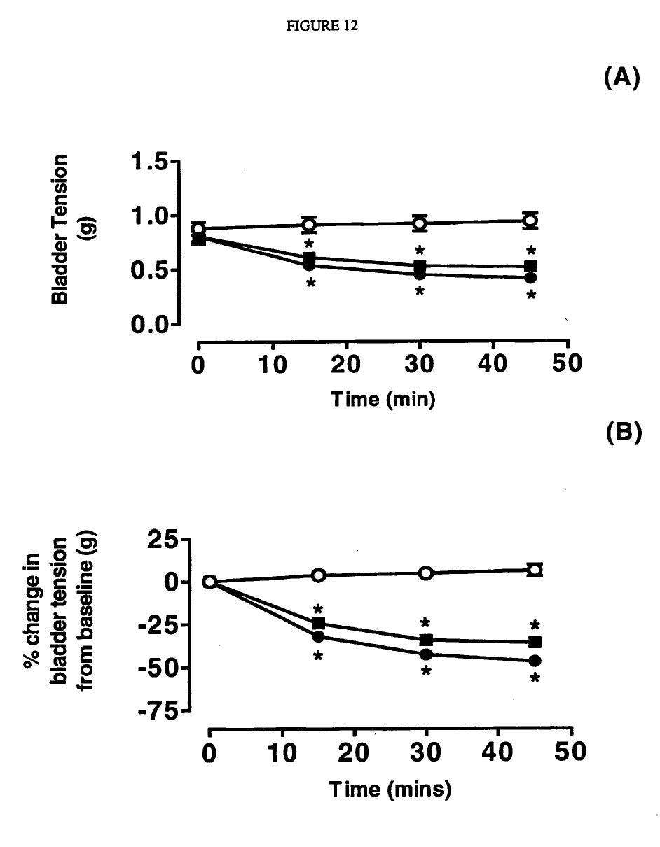

To further characterize the effects of Y-27632 on electrically-evoked bladder contractions, experiments were carried out using combinations of Y-27632, the muscarinic antagonist atropine and desensitization of P2X receptors with α,β-methylene ATP. Atropine (1 μM) had no effect on the amplitude of electrically-evoked bladder contractions at 2 and 4 Hz, but significantly attenuated these responses at 8 and 16 Hz (FIG. 11A (a); n=5). Furthermore, atropine significantly attenuated the AUC of these contractions at all frequencies of stimulation and to a significantly greater extent than the inhibitory effects of this antagonist on the amplitude of responses (FIG. 11A (b)). Y-27632 (10 μM) alone had a significantly greater attenuating effect on the amplitude on contractile responses to field stimulation than atropine alone (FIG. 11A (a)), but the effects of either Y-27632 or atropine on the AUC of these responses were not significantly different (FIG. 11A (b)). Concomitant incubation of rat urinary bladder strips with Y-27632 (10 μM) and atropine (1 gM) significantly attenuated both the amplitude and AUC of electrically-evoked bladder contractions at all frequencies of stimulation (FIG. 11A (a) & (b); n=4). Furthermore, the inhibitory effect of a combination of Y-27632 with atropine was significantly greater than the effects of Y-27632 or atropine alone, with the exception of the AUC of the contractile response to 2 Hz electrical stimulation with which no significant differences between these experimental groups was observed (FIG. 11A (a) & (b)).

Desensitization of P2X receptors with α,β-methylene ATP (10 μM) had no effect on the amplitude or AUC of electrically-evoked bladder contractions at 2 and 4 Hz, but significantly attenuated both parameters of these responses at 8 and 16 Hz (FIG. 1B (a) & (b); n=4). There were no significant differences between the effects of α,β-methylene ATP on the amplitude and AUC of electrically-evoked bladder contractile responses. Furthermore, the inhibitory effects of atropine alone on the AUC of these responses were significantly greater than the effects of α,β-methylene ATP alone, whereas no significant differences between the effects of these antagonists on the amplitude of electrically-evoked bladder contractions were observed (FIGS. 11A & B). The effects of Y-27632 (10 μM) alone on the amplitude of electrically-evoked bladder contractions were not significantly different from the effects of α,β-methylene ATP alone (FIG. 11B (a)), whereas Y-27632 had a significantly greater inhibitory effect on the AUC of these responses than α,β-methylene ATP (FIG. 11B (b)). Concomitant incubation of rat urinary bladder strips with Y-27632 (10 μM) and α,β-methylene ATP (10 μM) significantly attenuated the amplitude and AUC of electrically-evoked bladder contractions at all frequencies of stimulation (FIG. 11B (a) and (b); n=4). Furthermore, the inhibitory effects of a combination of Y-27632 with α,β-methylene ATP were significantly greater than the effects of Y-27632 or α,β-methylene ATP alone. In addition, the concomitant addition of Y-27632 and atropine to isolated rat urinary bladder strips had a greater attenuating effect on the AUC of electrically-evoked contractions than a combination of Y-27632 and α,β-methylene ATP, whereas no differences were observed between these two combinations of antagonists on the amplitude of these responses (FIGS. 11B & C). Frequency-response curves were abolished by further administration of TTX in all experimental groups (1 μM; n=26; unpublished data).

(5) Effects of Test Substances on Baseline Bladder Tensions

Y-27632 (10 μM) caused a significant decrease in baseline bladder tension during the 45 min incubation period, reaching a relaxation of 0.40±0.05 g, which amounted to a 47.0±2.5% decrease from baseline (n=22; FIG. 12). Similarly, HA-1077 (10 μM) evoked a significant decrease in baseline bladder tension, causing a 36.1±1.6% decrease, which was 0.30±0.01 g, after 45 min (n=5; FIG. 12). The effects of Y-27632 and HA-1077 on baseline bladder tensions were not significantly different. All other test substances used in the present study had no effect on baseline bladder tensions.

(6) Effects of Vehicle on Agonist- and Electrically-Evoked Bladder Contractions and Baseline Bladder Tensions

Administration of vehicle for test substances (distilled water) had no effect on agonist- or electrically-evoked contractions and baseline bladder tensions in all experimental groups.

All documents cited herein and patent applications to which priority is claimed are incorporated by reference herein in their entirety. This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

1. A method for modulating bladder smooth muscle activity comprising the step of contacting a polypeptide in a ROCK pathway with a compound that modulates an activity of said polypeptide.

2. The method of claim 1 wherein said polypeptide is ROCK 1 or ROCK 2 or both.

3. The method of claim 1 wherein said activity of said polypeptide is selected from the group consisting of:
a response to carbachol,
a response to neurokinin A,
a response to a P2X receptor antagonist, and
a response to electrical stimulus.

4. The method of claim 3 wherein said response is bladder contraction.

5. The method of claim 1 wherein said bladder smooth muscle activity is contractility.

6. A method of treating a mammal for a lower urinary tract disorder or overactive bladder comprising the step of modulating bladder smooth muscle activity.

7. A method of treating a mammal for a lower urinary tract disorder or overactive bladder comprising contacting said mammal with a compound that modulates an activity of a polypeptide in a ROCK pathway.

8. The method of claim 7 wherein said polypeptide is ROCK 1 or ROCK 2 or both.

9. The method of claim 7 wherein said activity of said polypeptide is selected from the group consisting of:
a response to carbachol,
a response to neurokinin A,
a response to a P2X receptor antagonist, and
a response to electrical stimulus.

10. The method of claim 9 wherein said response is bladder contraction.

11. The method of claim 6 wherein said bladder smooth muscle activity is contractility.

12. The method of claim 6 wherein said bladder smooth muscle activity is related to expression or activity of ROCK 1 or ROCK 2 or both.

13. The method of claim 12 wherein said activity of ROCK I or ROCK 2 or both is selected from the group consisting of:
a response to carbachol,
a response to neurokinin A,
a response to a P2X receptor antagonist, and
a response to electrical stimulus.

14. The method of claim 13 wherein said response is bladder contraction.

15. The method of claim 14 wherein said bladder smooth muscle activity is contractility.


SEQUENCE LISTING (6 sequences)

SEQ ID NO: 1 - DNA, 19 bp, Rattus rattus
aggcctgtgccaaaccttt

SEQ ID NO: 2 - DNA, 21 bp, Rattus rattus
tggtccctgtgggacttaaca

SEQ ID NO: 3 - DNA, 24 bp, Rattus rattus
ccgcctgccctagagtgtcgaaga

SEQ ID NO: 4 - DNA, 20 bp, Rattus rattus
cccgatcatcccctagaacc

SEQ ID NO: 5 - DNA, 20 bp, Rattus rattus
ttggagcaagctgtcgactg

SEQ ID NO: 6 - DNA, 25 bp, Rattus rattus
caacaaaaccagtccattcggcggc